(19) United States
(12) Patent Application Publication
(10) Pub. No.: US 2010/0106030 A1
(43) Pub. Date: Apr. 29, 2010

(54) METHOD AND SYSTEM FOR AUTOMATED MEASUREMENT OF PULSUS PARADOXUS

(76) Inventor: Gregory R. Mason, Manhattan Beach, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/604,346

(22) Filed: Oct. 22, 2009

Related U.S. Application Data
(60) Provisional application No. 61/107,974, filed on Oct. 23, 2008.

(51) Int. Cl. A61B 5/022

(52) U.S. Cl. 600/493

(57) ABSTRACT

A method and system for automatically detecting pulsus paradoxus. In one embodiment, the method includes automatically detecting pulsus paradoxus based upon a consideration of a blood pressure component, an audio component indicative of Korotkoff sounds and a respiratory component. The system includes a plurality of input modules for determining a blood pressure, a Korotkoff sound and a respiration of a subject. The system further includes a computing device coupled to the input modules for automatically determining a presence of pulsus paradoxus based on the blood pressure, the Korotkoff sound and the respiration of the subject.

### CROSS REFERENCE TO RELATED APPLICATION

This application claims priority from U.S. Provisional Patent Application Ser. No. 61/107,974, filed on Oct. 23, 2008.

### BACKGROUND

1. Field

A method and system for automated measurement of pulsus paradoxus.

2. Background

Pulsus paradoxus is a term referring to a systolic arterial pressure and pulse pressure that weakens abnormally during inspiration. It was first recognized in 1873 when an irregularity of the palpable pulse was observed while the heart sounds indicated that the cardiac rhythm was regular. It was found that the “irregularity” of the pulse resulted from a reduction in the absolute blood pressure in the extremity, leading to an impalpable pulse during inspiration. Pulsus paradoxus may be symptomatic of various abnormalities including pericardial tamponade, worsening asthma, chronic obstructive pulmonary disease, congestive heart failure, pulmonary edema, chronic dyspnea, obstructive sleep apnea and tension pneumothorax. If left undetected, these disorders may result in deterioration or death of critically ill patients. Thus, early detection is essential.

In a healthy individual, arterial and venous blood pressures vary throughout the respiratory cycle. It is not uncommon to see an increase in blood pressure during expiration followed by a decrease in blood pressure during inspiration. Such fluctuations may occur due to the intrathoracic pressure changes generated during breathing. Although the exact physiology behind pulsus paradoxus may vary depending upon the disease it is symptomatic of, the exaggerated pressure decrease may generally be explained by the interrelationship between the changing intrathoracic pressure during the respiratory cycle and the two ventricular chambers of the heart. During inspiration, intrathoracic pressure decreases which augments right heart filling, pulmonary vascular compliance and right ventricular stroke volume while reducing left heart filling and output.

Pulsus paradoxus may be detected by monitoring changes in blood pressure throughout the respiratory cycle. Under normal conditions, an individual may experience a decrease in arterial blood pressure of less than 10 millimeters mercury (mmHg) during inspiration. An abnormality is identified where this pressure decrease exceeds 10 mmHg. Currently there are a variety of techniques available for detecting this pressure decrease during inspiration.

One such technique for detection of pulsus paradoxus requires gradually deflating a sphygmomanometer (blood pressure cuff) while listening for the onset of Korotkoff sounds (sounds resulting from arterial pressure waves passing through the occluding cuff) during normal respiration. The Korotkoff sounds will first be audible during expiration only, and after further deflation of the cuff, during inspiration as well. If the cuff is deflated more than 10 mmHg between detection of intermittent and constant Korotkoff sounds, pulsus paradoxus is said to be present. This method of detection is problematic for a number of reasons, not the least of which is that automated blood pressure monitoring recording is now standard throughout the health care industry and this technique is incapable of detecting respiratory fluctuations in arterial pressure since only one set of systolic and diastolic pressures are recorded. Even when manual sphygmomanometers are available, the operator has to know how to perform the test to determine if pulsus paradoxus is present. In addition, blood pressure values are subjective in that they are reliant upon the operator's ability to listen to the sounds while watching the fluctuations of the gauge. The only record is therefore the operators' hand-written description of a highly subjective test.

A second technique used in detecting pulsus paradoxus is by direct monitoring of arterial pressure with an indwelling intra-arterial catheter. This technique is more accurate than sphygmomanometry in detecting pulsus paradoxus because it results in a permanent recording of the arterial waveform and pressure and thus allows for an objective measurement. Due to its invasive nature, however, it is often painful to the patient and requires a highly trained health care provider.

Infrared photosensors used for pulse oximetry and plethysmography may be utilized for a third technique that may be used for detecting pulsus paradoxus. In this technique, changes in the intensity of an infrared (IR) beam passing through a patient's finger tip, toe, or earlobe are obtained to measure fluctuations in regional blood volume, a correlation of blood pressure. A clip-on probe sends an IR beam through the fleshy tissue and receives reflections therefrom. Changes in the amount of blood in the measurement area (i.e., a capillary bed) cause changes in absorption or variation, and such changes vary along with the amount of blood delivered to that tissue. Thus, although not a direct measure of the patient's blood pressure, the plethysmographic signal emulates the waveform contour and magnitude of direct intra-arterial pulse pressure and is typically displayed on a monitor screen along with the electrocardiogram and respiratory excursions. Clinical use of this measurement, called plethysmographic oximetry (PO), has been reported to detect pulsus paradoxus in children with pericardial tamponade and in adults with respiratory distress from obstructive lung disease but these results have not yet been incorporated into devices that would make possible simply applied, non-invasive, real-time detection of pulsus paradoxus.

The above described techniques for manual detection of pulsus paradoxus are highly subjective, labor intensive and inaccurate (if measured with a blood pressure cuff), or invasive (if done with arterial lines) and therefore, are rarely used. Thus, the ability to detect the ominous condition of pulsus paradoxus requires a high level of suspicion and cumbersome or invasive technology. As a result, pulsus paradoxus and therefore early signs of several life-threatening conditions often go undetected.

### BRIEF DESCRIPTION OF THE DRAWINGS

The following illustration is by way of example and not by way of limitation in the figures of the accompanying drawings in which like references indicate like elements. It should be noted that references to “an” or “one” embodiment in this disclosure are not necessarily to the same embodiment, and such references mean at least one.

FIG. 1 illustrates an embodiment of a system for automatically detecting pulsus paradoxus.

FIG. 2 illustrates a display of the system of FIG. 1.

FIG. 3 illustrates the system of FIG. 1 used on a subject.

FIG. 4 illustrates a process flow diagram for automatically detecting pulsus paradoxus.

FIG. 5 illustrates a process flow diagram for automatically detecting pulsus paradoxus.

### DETAILED DESCRIPTION

Automatic and sensitive detection of pulsus paradoxus may be a major asset in emergency departments, medical and surgical intensive care units (ICU), operating rooms, and hospices since it is especially difficult to determine worsening of many conditions with current manual systems and those focusing on changes in arterial blood pressure alone. Such detection may allow health care providers to more quickly address and treat conditions such as, for example, cardiac tamponade and tension pneumothorax. Moreover, an automated system and method as described herein may be useful in nursing homes or hospices where detection of impending respiratory failure may allow families to be notified of important changes in conditions as they occur. It is thus believed, automated detection of pulsus paradoxus would help to identify and intervene in patient care resulting in thousands of lives saved each year and/or improved treatment regimen.

In one embodiment, a system for automatic analysis and detection of pulsus paradoxus is disclosed. In one embodiment, the system may include a measurement of blood pressure and Korotkoff sounds. In this aspect, the system may include leads connected to a blood pressure cuff and an electronic microphone or oscillometric device. The system may further include a lead including sensors for monitoring a patient's respiratory activity. In this aspect, the leads may include electrocardiogram sensors (ECG) associated with an impedance plethysmograph for detecting a respiration of the patient. The system may further optionally include leads having sensors for detecting a heartbeat or fluctuations in regional blood volume of the patient. The system may automatically process and analyze these measurements to determine the presence of pulsus paradoxus. If the presence of pulsus paradoxus is detected, the system may alert the health care physician by, for example, sounding an alarm or other similarly suitable alerting mechanism. The system may further record information relating to each of these measurements for future evaluation and display the information on an interface of the system for visual evaluation by a health care provider.

The system disclosed herein is an improvement over the current techniques currently employed by a health care provider to detect pulsus paradoxus. In particular, for manual detection of pulsus paradoxus, the health care provider attaches a blood pressure cuff to the patient. Once attached, the health care provider manually inflates the cuff to about 20 mmHg over the last measured systolic pressure. A pressure screw for deflation of the cuff is manually turned by the provider to slowly release the pressure. While watching the patient breath normally (without deep or shallow breaths), the provider listens for a pressure at which the first Korotkoff sound appears. While deflating the cuff slowly enough to capture at least three respiratory cycles, it is noted by the provider whether the first Korotkoff sound is immediately continuous or disappears with inspiration. If it does not disappear, it is concluded by the provider that pulsus paradoxus is not present. If, however, the Korotkoff sounds seem to the provider to be intermittent and disappear with inspiration, the provider continues deflation until the point were the Korotkoff sounds are continuous. The provider then notes these points and substracts the pressure at which the Korotkoff sounds first became continuous from the pressure at which the sounds were intermittent to confirm the presence of pulsus paradoxus. The results are then manually recorded in the medical record. Typically there is no visual record of these results or validation. In addition, the above steps need to be repeated several times to confirm the result. Thus, as can be seen from the manual system currently employed by providers, there is a high degree of subjectivity in the results and virtually no way to review or reproduce the results. As a result, the presence of pulsus paradoxus is generally perceived as difficult to diagnose in the health care industry and is often missed.

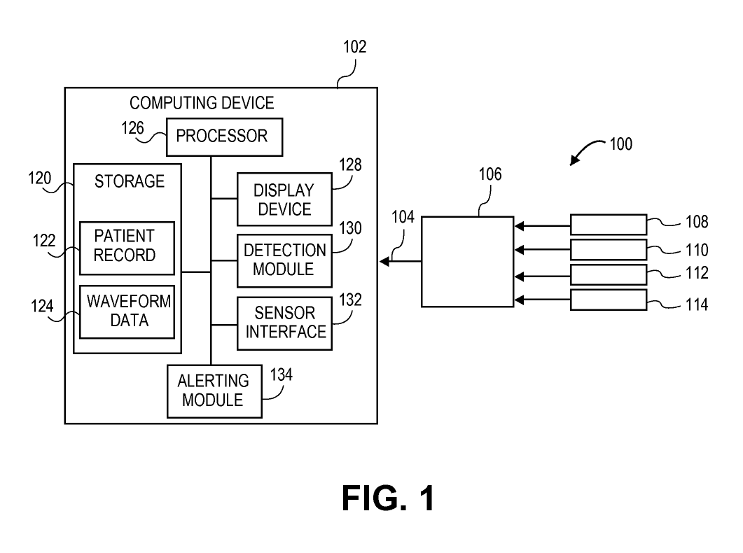

FIG. 1 illustrates a system for automatically detecting pulsus paradoxus. System 100 includes electronic device 102 for processing and displaying various system measurements. Electronic device 102 may be any computing device capable of performing program execution such as a desktop, laptop, handheld, server or other similarly suitable type of wired or wireless computing device. Device 102 may include a storage device 120 to store data over a period of time. In one embodiment, storage device 120 may store patient record information 122. Patient record information 122 may include, for example, waveform data 124 corresponding to the physiological status of the patient (e.g., plethysmographic data, respiratory data, etc.). Electronic device 102 may also include processor 126 for processing data associated with the patient and sensor interface 132 for transfer of data between electronic device 102 and a device sensor which receives measurement data and converts it to an electronic signal for processing. In some embodiments, electronic device 102 includes display device 128 for displaying physiological data associated with the patient. Electronic device 102 may also include detection module 130 for detecting a physiological condition of the patient (e.g. pulsus paradoxus) and alerting module 134 to, for example, alert a health care provider when the condition is detected.

Storage device 120 may further include a machine-readable storage medium (or more specifically a computer-readable storage medium) on which is stored one or more sets of instructions (e.g., for automated detection of pulsus paradoxus) embodying any one or more of the methodologies or functions described herein. During execution thereof by electronic device 102, processor 126 may also constitute machine-readable storage media.

The machine-readable storage medium may also be used to store the instructions for automated detection of pulsus paradoxus persistently. While the machine-readable storage medium is discussed in an exemplary embodiment to be a single medium, the term “machine-readable storage medium” and also “computer-readable medium” should be taken to include a single medium or multiple media (e.g., a centralized or distributed database, and/or associated caches and servers) that store the one or more sets of instructions. The terms “machine-readable storage medium” and “computer-readable medium” shall also be taken to include any medium that is capable of storing or encoding a set of instructions for execution by the machine and that cause the machine to perform any one or more of the methodologies of the present invention. The terms “machine-readable storage medium” and “computer-readable medium” shall accordingly be taken to include, but not be limited to, solid-state memories, and optical and magnetic media.

Device 102 may be associated with signal conditioner 106 which allows device 102 to record, process and display a wide range of measurements including voltage waveforms. In some embodiments, signal conditioner 106 is a PICOSCOPE® which may be connected to device 102 via USB port 104.

Input modules 108, 110, 112 and 114 may be connected to device 102. Input modules 108, 110, 112 and 114 may be modules which detect the physiological status of the patient and transmit such physiological data to device 102 for processing. The information may be input to device 102 through signal conditioner 106. Representatively, input module 108 may be a blood pressure cuff or sphygmomanometer for detecting a blood pressure of a patient. Input module 110 may be an audio device such as an electronic microphone or oscillometric device which detects Korotkoff sounds heard during measurement of the patient's blood pressure. Input module 112 may be a device for detecting a respiratory cycle of a patient such as an impedance plethysmograph, a capnograph or thermistor derived breaths. Input module 114 may be a device for measuring fluctuations in regional blood volume, a correlation of blood pressure. In this aspect, module 114 may be a pulse oximeter having a clip-on infrared probe or a plethysmograph. Although not a direct measure of the patient's blood pressure, the plethysmographic signal obtained by the pulse oximeter may emulate the waveform contour and magnitude of direct intra-arterial pulse pressure.

Although four modules are illustrated in FIG. 1, it is contemplated that fewer modules or additional modules (e.g., a module for detecting a patient's heart rhythm or electrocardiogram) may be associated with device 102. Although specific examples of modules 108, 110, 112 and 114 are discussed herein, it is contemplated that other similarly suitable devices as are known may be used to obtain the desired patient information.

To detect pulsus paradoxus using device 102, blood pressure cuff 108, which may be attached to electronic microphone (or oscillometric device) 110, is positioned, for example, on the arm of the patient. In this aspect, both the blood pressure and the Korotkoff sounds of the patient are detected. In addition, electrocardiogram leads of impedance plethysmograph 112 and pulse oximeter 114 may be attached to the patient for monitoring of the patient's respiration and oxygen saturation/pressure levels.

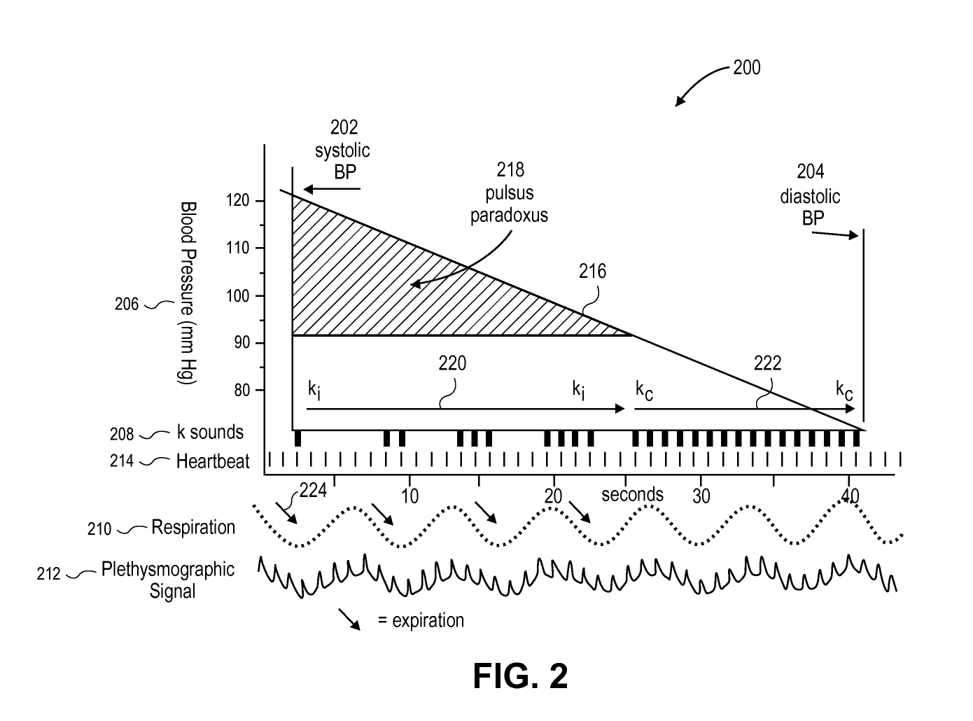

An actuation module associated with device 102 (or the blood pressure cuff) may be used to actuate one or more of modules 108, 110, 112 and 114. The actuation module may be, for example, a button, switch or key on a keyboard associated with device 102. Actuation of blood pressure cuff 108 causes it to automatically inflate and deflate. Blood pressure cuff 108 is slowly (e.g., 1-2 mmHg/sec) deflated to obtain a calibrated sphygmomanometer blood pressure. The first Korotkoff sounds representing systolic blood pressure are detected by electronic microphone 110 and transmitted to device 102 along with blood pressure information (e.g., a measurement of systolic blood pressure). In one embodiment, device 102 detects systolic blood pressure using blood flow oscillations (i.e. the pulse) detected by module 108 (e.g., blood pressure cuff). When pulsus paradoxus is present, the first Korotkoff sounds will be variable and alternately audible and inaudible, with inspiration initiating the inaudible portion. Thus, the first Korotkoff sounds are intermittent Korotkoff sounds (Ki). As the pressure of blood pressure cuff 108 is lowered, the Korotkoff sounds become continuous (Kc). Pulsus paradoxus is detected by subtracting the pressure at which Kc is heard from the pressure at which Ki is heard and occurs within ranges between 5 and 30 mmHg. Normal Ki ranges are from about 6 to 10 mmHg such that detection within a range above 10 mmHg is considered abnormal. This difference between Ki and Kc and the pressure range is automatically calculated by device 102 as will be described in more detail in reference to FIG. 2. Device 102 may include an alerting module which produces a sound or otherwise notifies a provider when a Ki pressure of equal to or greater than about 10 mmHg is calculated, or in another embodiment, when a Ki pressure equal to or greater than 12 mmHg is calculated. The alerting module may be, for example, an alarm or other device that produces an electronic signal that can be transmitted to a care station or to a physician (e.g., via a mobile device).

Additional plethysmographic signals (from the pulse oximeter) and respiratory movement are displayed on device 102 in the form of waves and allow for examination and confirmation of the measurements. Since each of these measurements are recorded and displayed on device 102, they can be immediately analyzed or printed and stored for later evaluation.

In the embodiment illustrated in FIG. 1, system 100 includes device 102 in which a waveform analysis may be implemented to determine pulsus paradoxus based on the previously discussed measurements (e.g. blood pressure and Korotkoff sounds). In another embodiment, at least a portion of the waveform data analysis may be performed manually without a computer system or software.

Measurement data for a patient stored in storage device 120 of device 102 may be processed by processor 126 running on device 102. In one embodiment, processor 126 may include a waveform analysis application which converts the waveform data into a suitable searchable format and stores the converted waveform data such that both the waveform data and the converted waveform data are stored in a patient's record. Each record will then contain waveform data suitable for providing waveform images as well as converted data suitable for searching and analysis.

There are a number of ways physiological data generated by modules 108, 110, 112 and 114 (e.g. waveform data) may be converted into a format that is searchable and will enable queries and calculations to be performed on the converted data. Waveform data may include a magnitude of Korotkoff sound or pulse data (volume/time), plethysmographic signals (or oximetry waveforms) and respiratory movement. In one implementation, at least a portion of the waveform data is converted into a text format by expressing the waveform data in terms of numerical values determined at a certain time interval. In another implementation, at least a portion of the waveform data is expressed in terms of a function (e.g., derivative) of waveform data at various time periods.

Alternatively or in addition to, pertinent features or patterns relating to the waveform data are detected and stored in the corresponding patient record to facilitate subsequent searching and/or calculations. In one embodiment, portions of the waveform data are examined by the waveform analysis application to extract features or patterns that are pertinent to analysis of the waveform data. For example, the waveform analysis application may be configured to recognize certain conditions indicative of pulsus paradoxus (e.g., intermittent Korotkoff sounds within a pressure range above 10 mmHg) by examining relevant data and when such condition is detected, the waveform analysis application may write an entry in, for example, a corresponding patient record indicating the occurrence of such condition and when it occurred. Extracting of pertinent features or patterns of the waveform data can also be accomplished by expressing a function (e.g., derivative) in terms of time and denote specific high points or low points or changes of directions.

There are a number of other techniques that may be used to extract pertinent information from the waveform data. For example, pertinent information from the waveform data can be extracted by determining frequency and amplitude of the waveform at various points. The waveform data can also be analyzed by examining each cycle of the waveform, individually. This may be accomplished by capturing a segment of the waveform data that defines a single cycle and analyzing the captured segment, perhaps by applying a suitable algorithm, such as pattern recognition algorithm or transform algorithm.

FIG. 2 illustrates a display of the device of FIG. 1. FIG. 2 represents display 200 of device 102 when abnormal pulsus paradoxus is present. Blood Pressure (BP) 206 and time (seconds) are on the ordinate and abscissa axes, respectively. In this embodiment, five signals obtained from modules such as those discussed in reference to FIG. 1 are measured and displayed on device 102. The signals correspond to blood pressure 206, Korotkoff sounds 208, respiration 210, plethysmographic signal 212 and optional heartbeat or pulse 214 measurements.

Blood pressure 206 is indicated by diagonal line 216 representing a BP measurement from systolic BP 202 to diastolic BP 204. Korotkoff sounds 208 and their frequency are illustrated as thick black vertical lines along the abscissa axis. Intermittent Korotkoff sounds 220 are labeled as Ki and continuous Korotkoff sounds 222 are labeled as Kc. Respiration 210 is illustrated as a waveform with expiration indicated by downward arrow 224. Plethysmographic signal 212 is further illustrated as a waveform. Although heartbeat 214 is displayed, it is also represented in the peaks of the plethysmographic waveform and is therefore optional.

As previously discussed, diagonal line 216 represents the measured BP 206, from systolic BP 202 to diastolic BP 204, when Ki=Kc and the difference of Ki−Kc=0. When normal pulsus paradoxus is present, shaded area 218, defined by intermittent Ki−Kc is usually less than 10 mmHg. When abnormal pulsus paradoxus is present, Ki−Kc is greater than 10 mmHg. In FIG. 2, pulsus paradoxus is determined as follows: 122 mmHg (first Ki sounds)−92 mmHg (first Kc sounds)=30 mmHg. Thus since Ki−Kc is greater than 10 mmHg, abnormal pulsus paradoxus is determined. Device 102 alarm then signals an alert that abnormal pulsus paradoxus may be present. It is noted that the Korotkoff sound corresponds to a heartbeat but the converse does not. Based upon the input from modules 108, 110, 112 and 114 as previously discussed, device 102 is able to automatically detect and record each of the above measurements and analyze the data to determine whether pulsus paradoxus is present. It is noted that only the data relating to the blood pressure and Korotkoff sounds of the patient are necessary for the device to perform the above-described calculation. Since the measurements can further be recorded by device 102, they are available for later review and evaluation by a health care provider.

Pulsus paradoxus can also be seen through careful examination of Ki as displayed on device 102. In particular, during Ki, Korotkoff sounds can be seen to disappear during inspiration such that they are present only during expiration. As pressure declines, Korotkoff sounds are more and more numerous until they appear during both exhalation and inhalation (i.e., Kc). This disappearance of Korotkoff sounds during inspiration suggests the presence of pulsus paradoxus. In some embodiments, device 102 is programmed to alert (e.g. alarm) a health care provider when such conditions are met.

Additional indicators of pulsus paradoxus can be found upon review of plethysmographic signal or waveform 212. Plethysmographic waveform 212 normally does not oscillate significantly from the baseline with respiration. When pulsus paradoxus is present, however, plethysmographic waveform 212 oscillates markedly as seen in FIG. 2. This variation is the exact same cycle length as respiratory frequency illustrated by respiratory waveform 210 but is usually offset slightly (about 10% of cycle length). These waveforms may then be analyzed and assigned a score by dividing averaged offsets of each pulse wave (i.e., waves of oximetry waveform 212) from a base line with average wave amplitudes over a respiratory cycle. A score falling within a particular range indicates an abnormality (e.g. oscillating base of waveform 212 from a baseline). In one embodiment, the range indicative of abnormal pulsus paradoxus may be, for example, a score from about 0.3 to about 2.5. Thus, in another embodiment, device 102 may be programmed to detect a threshold oscillation in waveform 212 above which is an indicator of pulsus paradoxus.

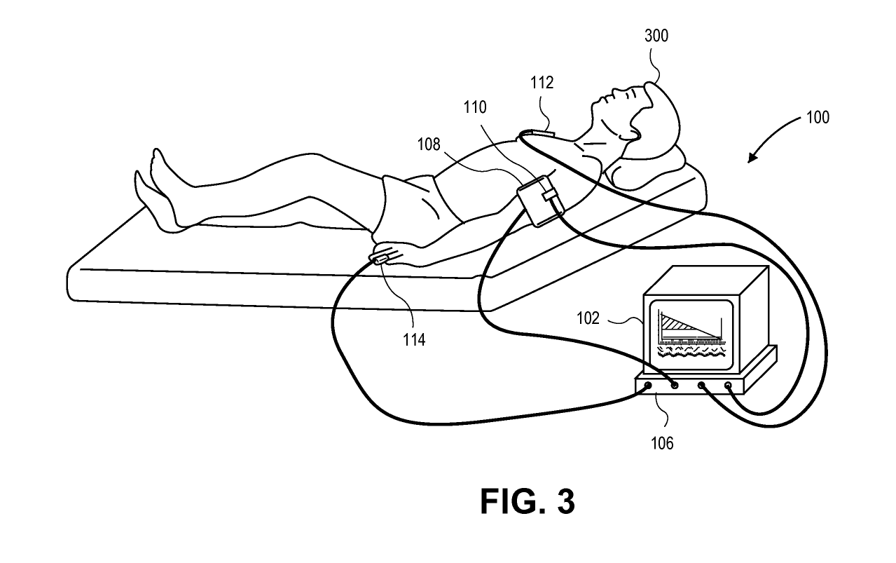

FIG. 3 illustrates the system of FIG. 1 used on a subject. System 100 includes electronic device 102 for processing and displaying various system measurements and signal conditioner 106 as discussed in reference to FIG. 1. Input modules 108, 110, 112 and 114 are connected to device 102. In this embodiment, input module 108 may be a blood pressure cuff positioned around an arm of subject 300. Input module 110 may be an audio device such as an electronic microphone attached to blood pressure cuff 108. Input module 110 detects Korotkoff sounds of subject 300. Input module 112 is positioned on a chest of subject 300. Input module 112 may be a device for detecting a respiratory cycle of a patient, for example, an impedance plethysmograph. Input module 114 is further positioned on subject 300 and detects fluctuations in regional blood volume. In this aspect, module 114 may be a pulse oximeter having a clip-on infrared probe attached to a finger of subject 300.

To detect pulsus paradoxus of subject 300, the health care provider switches an actuation module (not shown) of device 102 to an “on” position. Modules 108, 110, 112 and 114 then begin measuring the blood pressure, respiration and oxygen saturation/pressure levels and recording the Korotkoff sounds of subject 300. Each of these measurements are then processed by signal conditioner 106 and displayed on device 102 for evaluation by the health care provider.

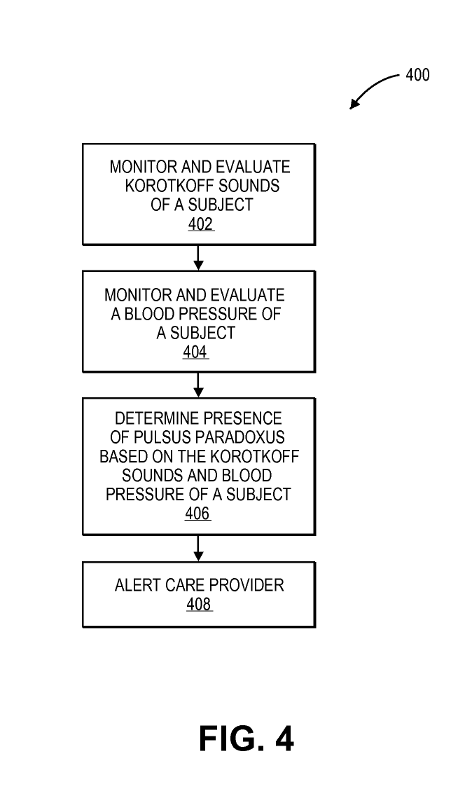

FIG. 4 illustrates a process for automatically detecting pulsus paradoxus. Process 400 includes monitoring and evaluating Korotkoff sounds of a subject (block 402). The Korotkoff sounds are monitored to determine the presence of intermittent Korotkoff (Ki) sounds and continuous Korotkoff (Kc) sounds as previously discussed. Process 400 further includes monitoring and evaluating a blood pressure of a subject (block 404). The presence of pulsus paradoxus is determined based on the Korotkoff sounds and blood pressure measurements of the subject (block 406) as discussed in reference to FIG. 2. In particular, as previously discussed, if a difference between the blood pressure at which first Ki sounds occur and the pressure at which first Kc sounds occur is greater than about 10 mmHg, pulsus paradoxus is present. If a determination is made that pulsus paradoxus is present, the health care provider is automatically alerted (block 408).

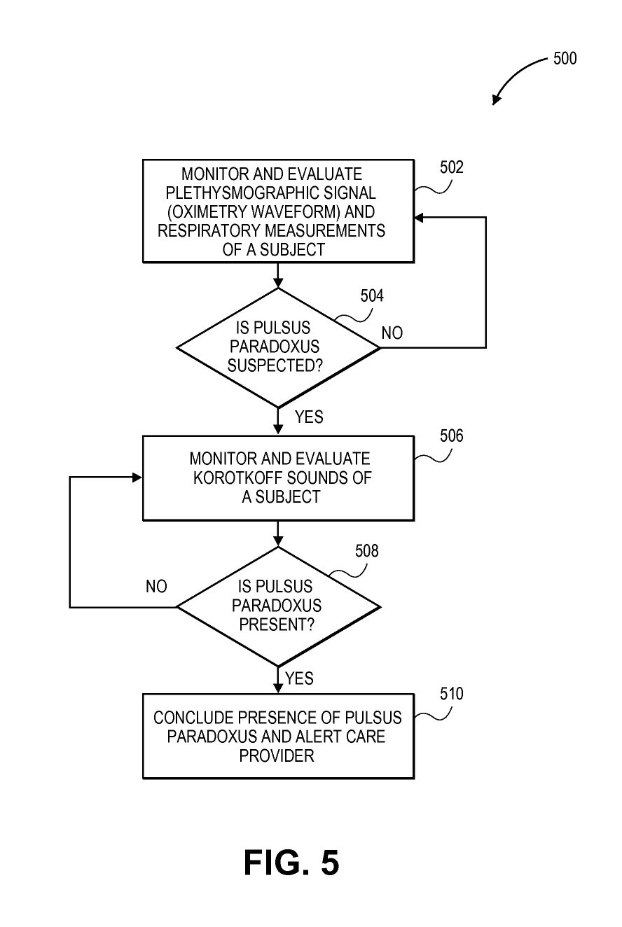

FIG. 5 illustrates a process flow diagram for a system for automatically detecting pulsus paradoxus. In one embodiment, process 500 includes the optional steps of monitoring and evaluating plethysmographic signals or waveform (i.e. waveform 212) and respiratory measurements of a subject (block 502). A determination is then made based on the plethysmographic waveform and respiratory measurements whether pulsus paradoxus is suspected (block 504). If a threshold oscillation of the plethysmographic waveform from a baseline is detected as previously discussed, pulsus paradoxus is suspected. If the threshold oscillation is not met, pulsus paradoxus is not suspected and the optional step of monitoring of the plethysmographic signal and respiratory measurements (block 502) continues. Process 500 further includes monitoring and evaluation of Korotkoff sounds of a subject (block 506). Based on such monitoring and evaluation, a determination is made as to whether pulsus paradoxus is present (block 508). In one embodiment, pulsus paradoxus is determined to be present if the difference between a pressure at which continuous Korotkoff sounds and intermittent Korotkoff sounds are present is above at least 10 mmHg. If the pressure is below 10 mmHg, it is determined that pulsus paradoxus is not present and monitoring of the Korotkoff sounds continues. When the Korotkoff sounds and (optional plethysmographic and respiratory data) indicate pulsus paradoxus is present, it is concluded that pulsus paradoxus is present and the system alerts the care provider (block 510). An alert may be an audible signal on device 102, an electronic signal on device 102 that can be transmitted to a care station or to a physician (e.g., via a mobile device). Thus, according to process 500, the presence of pulsus paradoxus may be initially suspected by monitoring the plethysmographic signal and respiratory measurements of a subject and then confirmed by evaluating the Korotkoff sounds of the subject.

A device, such as device 102, for performing the operations herein may be specially constructed for the required purposes or it may comprise a general purpose computer selectively activated or reconfigured by a computer program stored in the computer. Such a computer program may be stored in a computer readable storage medium, such as, but not limited to, any type of disk including floppy disks, optical disks, CD-ROMs and magnetic-optical disks, read-only memories (ROMs), random access memories (RAMs), EPROMs, EEPROMs, magnetic or optical cards, Flash memory devices including universal serial bus (USB) storage devices (e.g., USB key devices) or any type of media suitable for storing electronic instructions, each of which may be coupled to a computer system bus.

The algorithms and displays presented herein are not inherently related to any particular computer or other apparatus. Various general purpose systems may be used with programs in accordance with the teachings herein or it may prove convenient to construct a more specialized device to perform the described method. In addition, the invention is not described with reference to any particular programming language. It will be appreciated that a variety of programming languages may be used to implement the teachings of the invention as described herein.

A computer readable medium includes any mechanism for storing information in a form readable by a computer. For example, a computer readable medium includes read only memory (“ROM”), random access memory (“RAM”), magnetic disk storage media; optical storage media, flash memory devices or other type of machine-accessible storage media.

In the preceding detailed description, specific embodiments are described. It will, however, be evident that various modifications and changes may be made thereto without departing from the broader spirit and scope of the claims. The specification and drawings are, accordingly, is to be regarded in an illustrative rather than restrictive sense.

What is claimed is:

1. A method comprising:
automatically detecting by a computing device pulsus paradoxus based upon a consideration of a blood pressure and a Korotkoff sound of a subject.

2. The method of claim 1, wherein automatically detecting further comprises considering a respiratory cycle of the patient.

3. The method of claim 1, wherein automatically detecting further comprises considering fluctuations in regional blood volume of the subject.

4. The method of claim 1, wherein automatically detecting comprises:
determining a first blood pressure at which intermittent Korotkoff sounds occur;
determining a second blood pressure at which continuous Korotkoff sounds occur; and
determining whether a difference between the first blood pressure and the second blood pressure is at least 10 mmHg.

5. The method of claim 1, further comprising:
detecting pulsus paradoxus by detecting a disappearance of Korotkoff sounds during inspiration of the subject.

6. The method of claim 1, further comprising:
comparing an oscillation of a plethysmographic waveform from a baseline to a respiratory cycle length.

7. The method of claim 1, further comprising engaging an alarm when pulsus paradoxus is detected.

8. A system comprising:
a first module for determining a blood pressure of a subject;
a second module for differentiating intermittent and continuous Korotkoff sounds of the subject; and
a computing device coupled to the first module and the second module for automatically determining a presence of pulsus paradoxus based on the blood pressure and the Korotkoff sounds of the subject.

9. The system of claim 8, further comprising:
a third module coupled to the computing device for determining a respiratory cycle of the subject.

10. The system of claim 8, further comprising:
a fourth module coupled to the computing device for determining fluctuations in regional blood volume of the subject.

11. The system of claim 8, wherein the computing device comprises a signal conditioning device to process data from the first module and the second module.

12. The system of claim 8, wherein the computing device comprises a display capable of displaying for viewing information associated with the blood pressure and Korotkoff sounds of the subject.

13. The system of claim 8, wherein the computing device comprises an alerting module to automatically notify a health care provider of the presence of pulsus paradoxus.

14. A machine readable medium containing a set of instructions that when executed cause a computing device to perform a method comprising:
automatically detecting by a computing device pulsus paradoxus based upon a consideration of a blood pressure and a Korotkoff sound of a subject.

15. The machine readable medium of claim 14, wherein automatically detecting further comprises considering a respiratory cycle of the patient.

16. The machine readable medium of claim 14, wherein automatically detecting further comprises considering fluctuations in regional blood volume of the subject.

17. The machine readable medium of claim 14, wherein automatically detecting comprises:
determining a first blood pressure at which intermittent Korotkoff sounds occur;
determining a second blood pressure at which continuous Korotkoff sounds occur; and
determining whether a difference between the first blood pressure and the second blood pressure is at least 10 mmHg.

18. The machine readable medium of claim 14, wherein the method further comprises:
detecting pulsus paradoxus by detecting a disappearance of Korotkoff sounds during inspiration of the subject.

19. The machine readable medium of claim 14, wherein the method further comprises:
comparing an oscillation of a plethysmographic waveform from a baseline to a respiratory cycle length.

20. The machine readable medium of claim 14, further comprising engaging an alarm when pulsus paradoxus is detected.
